(19) United States
(12) Patent Application Publication
(10) Pub. No.: US 2026/0341659 A1
(43) Pub. Date: Oct. 29, 2026

(54) SYSTEM, METHOD AND MEDIUM FOR RAPID IDENTIFICATION OF EDIBLE OILS

(71) Applicant: The Hong Kong Polytechnic University, Kowloon (HK)

(72) Inventors: Zhongping Yao, Kowloon (HK); Ho Yin Michael Ma, Kowloon (HK); Suying Li, Kowloon (HK)

(73) Assignee: The Hong Kong Polytechnic University, Kowloon (HK)

(21) Appl. No.: 19/137,896

(22) PCT Filed: Apr. 23, 2025
(86) PCT No.: PCT/CN2025/090677

§ 371 Date: Jun. 11, 2025

(51) Int. Cl. H01J 49/00; G06N 20/00; H01J 49/16

(52) U.S. Cl. CPC H01J 49/0036; G06N 20/00; H01J 49/164

(57) ABSTRACT

Disclosing a method of identifying an unknown edible oil sample. The method comprises generating a hierarchical library of known edible oil samples; determining using matrix-assisted laser desorption/ionization mass spectrometry (MALDI-MS) spectral data including at least one or more TAG peaks for an unknown edible oil sample, training a machine learning module to identify a presumptive single edible oil type for unknown edible oil samples. The hierarchical library is generated by classifying a plurality of edible oil samples having known types into a predetermined number of primary representative groups according to intensity of one or more characteristic TAG peaks for that group using PCA analysis; further decomposing the primary representative groups using a binary tree classification network. Each level of the binary tree includes two members and the terminal level of the hierarchical library has two or more known edible oil members.

### FIELD OF THE DISCLOSURE

The present disclosure relates to a method, system and computer readable medium for identifying an unknown edible oil sample, in particular for rapidly identifying an unknown edible oil sample.

### BACKGROUND OF THE DISCLOSURE

Rapid and accurate methods for edible oil analysis are crucial in addressing oil adulteration issues. Due to the complex fatty acid composition of edible oils, detecting adulterants in edible oils is a challenging task, particularly when analyzing mixtures containing both unconventional oils (such as pistachio oil) and traditional oils (such as soybean oil).

Gas Chromatography-Flame Ionization Detection (GC-FID) is a widely adopted standard method for edible oil analysis. However, this method has several limitations: it requires lengthy column separation, laborious chemical derivatization, and skilled technicians to undertake complex data analysis. Additionally, multiple manual data entry and result interpretation steps are needed for oil sample testing, making high-throughput analysis difficult (International Organization for Standardization, 2015). Furthermore, analyzing GC chromatograms can be challenging because esterification leads to the loss of triacylglycerol (TAG) information, resulting in highly similar fatty acid profiles and reduced accuracy in oil classification. For example, the abundance ranges of oleic and linoleic acids in corn oil (20% to 42.2% and 34% to 65.6%, respectively) overlap with those specified in the Codex Alimentarius (2015) for grape seed oil (12% to 28% and 58% to 78%) and sesame oil (34.4% to 45.5% and 36.9% to 47.9%).

Other methods are developed, such as electronic nose, mid-infrared spectroscopy, Raman spectroscopy, and Matrix-Assisted Laser Desorption/Ionization (MALDI) mass spectrometry. These methods directly measure the chemical properties of edible oil samples so as to overcome the limitations of GC-FID. For example, MALDI mass spectrometry can directly determine the TAG profile of samples. Unknown samples can be identified by comparing their TAG profiles with those unique to specific edible oils (Lay et al., 2006). However, MALDI mass spectrometry data has high dimensionality, presenting classification challenges.

A PLS-DA (Partial Least Squares Discriminant Analysis) model is established for database-based classification using MALDI spectrometry data. However, this model is not suitable for multi-class classification with uneven sample sizes in the database.

Furthermore, the discriminant ability of PLS-DA models is limited, and such models can only accurately classify less common edible oils such as hazelnut oil (50% accuracy) and avocado oil (67.7% accuracy). Additionally, the predicted scores generated by PLS-DA models do not reflect the probability of the predicted class, leading to poor identification results for unknown edible oil samples being analysed.

Another approach which has been attempted is to combine PLS-DA models with cosine similarity scores. Unfortunately, this type of edible oil classification system relies on offline methods (in particular manual data binning and standardization of original MALDI mass spectrometry data, as well as manual input of data for analysis). Unfortunately, the manual nature of such offline methods hinders the use of MALDI mass spectrometry for high-throughput edible oil analysis.

It is an object of the present disclosure to address or at least partially ameliorate some of the above problems of the current approaches.

### SUMMARY OF THE DISCLOSURE

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

According to a first aspect of the present disclosure, there is a disclosed a method of identifying an unknown edible oil sample.

The method may comprise generating a hierarchical library of known edible oil samples, by classifying a plurality of edible oil samples having known types into a predetermined number of primary representative groups according to intensity of one or more characteristic TAG peaks for that group using PCA analysis, further decomposing the primary representative groups using a binary tree classification network, wherein each level of said binary tree includes two members, said two members being either intermediate representative groups comprising edible oil types grouped by PCA analysis of the TAG peaks or a single edible oil type defined using a determined maxima function of the TAG peaks of the known edible oil members therein; determining using matrix-assisted laser desorption/ionization mass spectrometry (MALDI-MS) spectral data including at least one or more TAG peaks for an unknown edible oil sample; and training a machine learning module to: (a) compare the obtained spectral data with TAG peaks for said unknown edible oil sample against the characteristic TAG peaks in each primary representative group of the hierarchical library of edible oil samples by calculating the similarity score between each primary representative group and the unknown edible oil sample; (b) select the primary representative group with the highest similarity score from the plurality of primary representative groups; and (c) compare the obtained spectral data with TAG peaks for said unknown edible oil sample by determining the similarity score between the obtained spectral data with TAG peaks for said unknown edible oil with the characteristic TAG peaks of the respective intermediate group; or the characteristic TAG peaks of the single representative oil type; and selecting either the intermediate group member or single representative edible oil type with the highest similarity score; and (d) repeating step (c) above until a presumptive single edible oil type for the unknown edible oil sample is identified.

Optionally, the obtained spectral data with TAG peaks for said unknown edible oil sample and the spectral data of the hierarchical library of edible oil samples may be normalized.

Optionally, the hierarchical library of known edible oil samples may have eight representative groups. The said eight representative groups may comprise edible oils with TAG peaks of a first group comprising 659.6, 660.6, 687.6, 688.6; TAG peaks of a second group comprising 895.7, 897.7, 899.7, TAG peaks of a third group comprising 853.7, 855.7, 879.8, 880.8, 881.8, TAG peaks of a fourth group comprising 880.8, 881.8, 903.8, 904.8, 905.8, 906.8, 907.8, 908.7, TAG peaks of a fifth group comprising 901.8, 902.8, 903.8, 904.8, 905.8, 906.8, 907.8, TAG peaks of a six group comprising 877.8, 878.8, 879.8, 880.8, TAG peaks of a seventh group comprising 853.7, 854.7, 879.8, 880.8, 881.8, 882.8, 905.8, 906.8, 907.8, 908.7, 909.7, TAG peaks of a eighth group comprising 735.3, 806.6, 828.6, 927.8, 955.7, 956.7, 957.7, 981.7, 1019.5, 1045.5

Optionally, the similarity scores may be calculated based on Gaussian mixture algorithm or Logistic regression algorithm.

According to a second aspect of the present disclosure, there is a system for identifying an unknown edible oil sample.

The system may comprise: a data store comprising a hierarchical library of known edible oil samples, said library including a plurality of edible oil samples classified into known types into a predetermined number of primary representative groups according to intensity of one or more characteristic TAG peaks for that group using PCA analysis, wherein said primary representative groups are further decomposed using a binary tree classification network, wherein each level of said binary tree includes two members, said two members being either intermediate representative groups comprising edible oil types grouped by PCA analysis of the TAG peaks or a single edible oil type defined using a determined maxima function of the TAG peaks of the known edible oil members therein and the terminal level of the hierarchical library has two or more known edible oil members; a processor configured for comparing the matrix-assisted laser desorption/ionization mass spectrometry (MALDI-MS) spectral data including at least one or more TAG peaks for an unknown edible oil sample against the characteristic TAG peaks in each primary representative group of the hierarchical library of edible oil samples by (a) comparing the obtained spectral data with TAG peaks for said unknown edible oil sample against the characteristic TAG peaks in each primary representative group of the hierarchical library of edible oil samples by calculating the similarity score between each primary representative group and the unknown edible oil sample; (b) selecting the primary representative group with the highest similarity score from the plurality of primary representative groups; and (c) comparing the obtained spectral data with TAG peaks for said unknown edible oil sample by determining the similarity score between the obtained spectral data with TAG peaks for said unknown edible oil with the characteristic TAG peaks of the respective intermediate group; or the characteristic TAG peaks of the single representative oil type; and selecting either the intermediate group member or single representative edible oil type with the highest similarity score; (d) repeating step (c) above until a presumptive single edible oil type for the unknown edible oil sample is identified.

According to a third aspect of the present disclosure, there is a computer program product for identifying an unknown edible oil sample which when executed by a processor causes it to perform the method according to the first aspect of the present disclosure.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended Figures. Understanding that these Figures depict only exemplary embodiments of the disclosure and are not therefore to be considered to be limiting of its scope, the principles herein are described and explained with additional specificity and detail through the use of the accompanying Figures.

Preferred embodiments of the present disclosure will be explained in further detail below by way of examples and with reference to the accompanying Figures, in which:—

FIG. 1 depicts an exemplary system according to an embodiment of the present disclosure.

FIG. 2 depicts a schematic representation of an exemplary hierarchical library of known edible oil samples according to an embodiment of the present disclosure.

FIG. 3 depicts characteristic peaks used for grouping of edible oil types in the primary representative level of the hierarchical library of FIG. 2.

FIG. 4 depicts exemplary TAG peaks used for grouping in the primary representative level and additional levels of a binary tree classification model used for the hierarchical library of FIG. 2.

FIG. 5a to FIG. 5o depict exemplary score plots obtained by Principal Component Analysis used for grouping in the primary representative level and additional levels of a binary tree classification model used for the hierarchical library of FIG. 2.

FIG. 6 depicts an exemplary schematic representation of a series of tree-based binary classifications of edible oil samples based on the decision of maxima function to calculated probabilistic score for the classification of samples according to the present disclosure.

FIG. 7 depicts an exemplary simplified flow chart of the method of identifying an unknown edible oil sample according to an embodiment of the present disclosure.

FIG. 8a depicts performance metrics for the system in identifying an unknown edible oil sample according to an embodiment of the present disclosure by calculating average accuracy, sensitivity, precision, and F1-score regarding validation dataset.

FIG. 8b and FIG. 8c depict the visualized performance of the system of identifying an unknown edible oil sample of the present disclosure based on confusion matrix regarding validation dataset.

FIG. 8d depicts identification results for the system in identifying an unknown edible oil sample according to an embodiment of the present disclosure for a testing dataset.

FIG. 9 depicts an exemplary user interface for an embodiment of the system of the present disclosure for identifying an unknown edible oil sample.

### DETAILED DESCRIPTION OF THE PREFERRED EMBODIMENTS

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure.

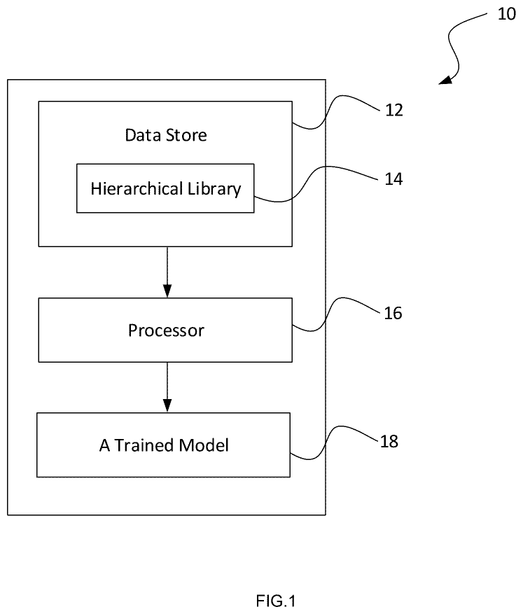

Referring to FIG. 1, there is shown a schematic representation of a system 10 for identifying an unknown edible oil sample.

The system 10 for identifying an unknown edible oil sample may comprise a data store 12. In the embodiment depicted, the data store 12 comprises a hierarchical library 14 of known edible oil samples. As depicted, the hierarchical library 14 includes a plurality of edible oil samples clustered into known types as a predetermined number of primary/intermediate representative groups according to intensity of one or more characteristic TAG peaks for that group using Principal Component Analysis (PCA).

The system 10 further includes a processor 16 configured for comparing the matrix-assisted laser desorption/ionization mass spectrometry (MALDI-MS) spectral data including at least one or more TAG peaks for an unknown edible oil sample against the characteristic TAG peaks in each primary representative group of the hierarchical library of edible oil samples as described herein.

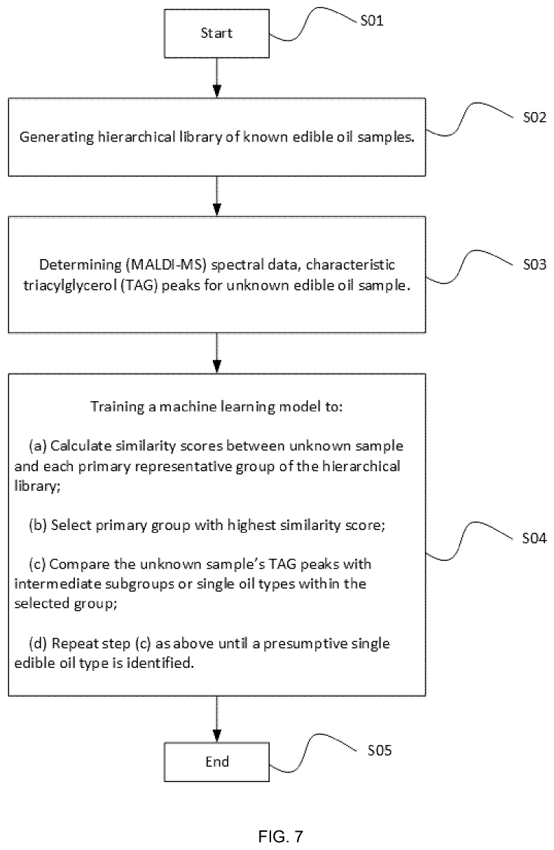

The system 10 further includes a machine learning module 18 which as trained as set out in S04 in FIG. 7 and described below.

The machine learning module 18 is trained to: (a) compare the obtained spectral data with TAG peaks for said unknown edible oil sample against the characteristic TAG peaks in each primary representative group of the hierarchical library of edible oil samples by calculating the similarity score between each primary representative group and the unknown edible oil sample; (b) select the primary representative group with the highest similarity score from the plurality of primary representative groups; and (c) compare the obtained spectral data with TAG peaks for said unknown edible oil sample by determining the similarity score between the obtained spectral data with TAG peaks for said unknown edible oil with the characteristic TAG peaks of the respective intermediate group; or the characteristic TAG peaks of the single representative oil type; and selecting either the intermediate group member or single representative edible oil type with the highest similarity score; (d) repeat step (c) above until a presumptive single edible oil type for the unknown edible oil sample is identified.

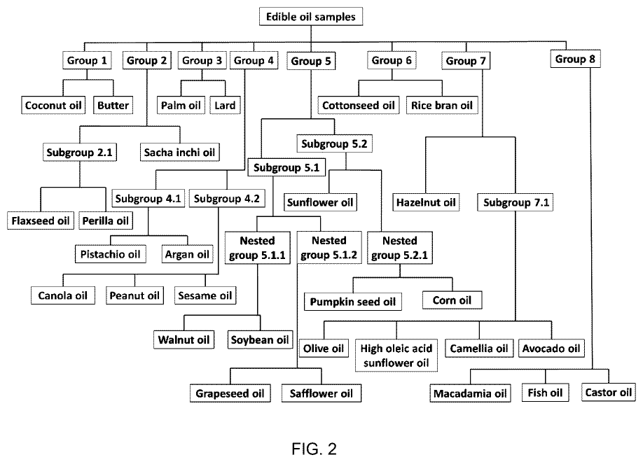

FIG. 2 Illustrates an example of the grouping of edible oil types in the hierarchical library 14.

In the embodiment as shown in FIG. 2, a series of Principal Component Analysis may be conducted for obtaining probabilistic scores for grouping in the primary representative level and additional levels of the hierarchical library 14. The samples are categorized into eight primary representative groups, i.e., Group 1, Group 2, Group 3, Group 4, Group 5, Group 6, Group 7 and Group 8, according to characteristics mass spectral patterns (TAG peaks) of the samples.

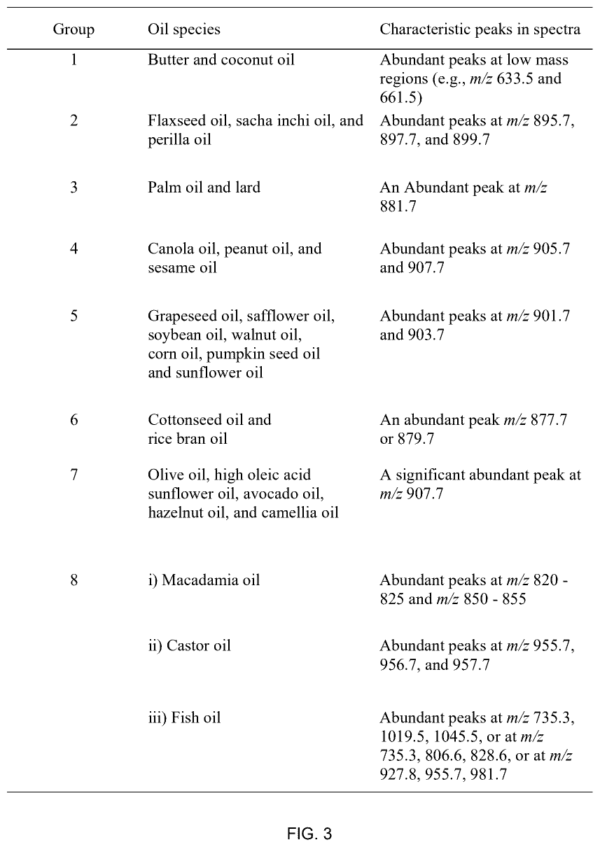

The characteristic peaks of edible oil types used for grouping in the primary representative level of the hierarchical library 14 is shown in FIG. 3.

The primary representative groups of the hierarchical library 14 may be further decomposed based on binary tree classification algorithm and according to Principal Component Analysis

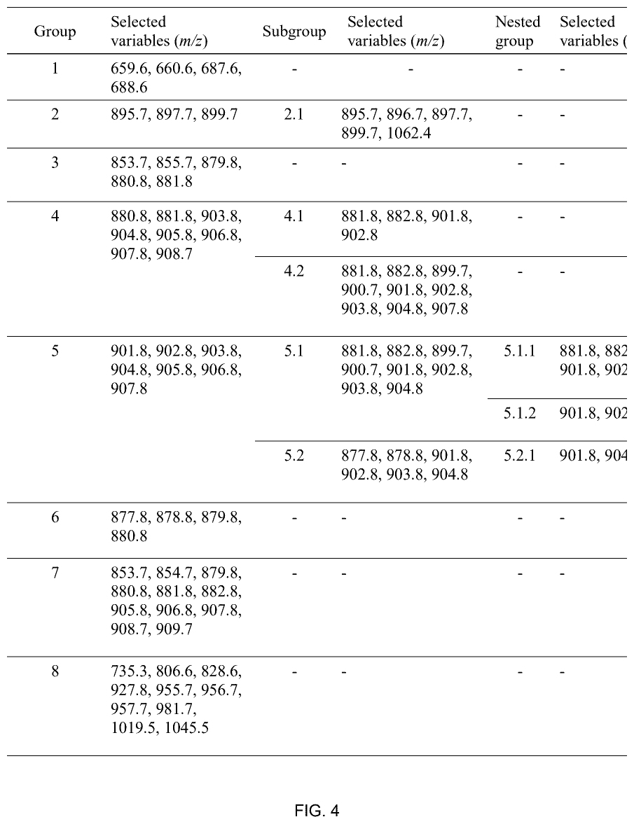

FIG. 4 illustrates an example of the grouping of TAG peaks in the hierarchical library 14.

As shown, Group 1 comprises TAG peaks of 659.6, 660.6, 687.6, 688.6; Group 2 comprises TAG peaks of 895.7, 897.7, 899.7; Group 3 comprises TAG peaks of 853.7, 855.7, 879.8, 880.8, 881.8, Group 4 comprises TAG peaks of 880.8, 881.8, 903.8, 904.8, 905.8, 906.8, 907.8, 908.7; Group 5 comprises TAG peaks of 901.8, 902.8, 903.8, 904.8, 905.8, 906.8, 907.8; Group 6 comprises TAG peaks of 877.8, 878.8, 879.8, 880.8; Group 7 comprises TAG peaks of 853.7, 854.7, 879.8, 880.8, 881.8, 882.8, 905.8, 906.8, 907.8, 908.7, 909.7; and Group 8 comprises TAG peaks of 735.3, 806.6, 828.6, 927.8, 955.7, 956.7, 957.7, 981.7, 1019.5, 1045.5.

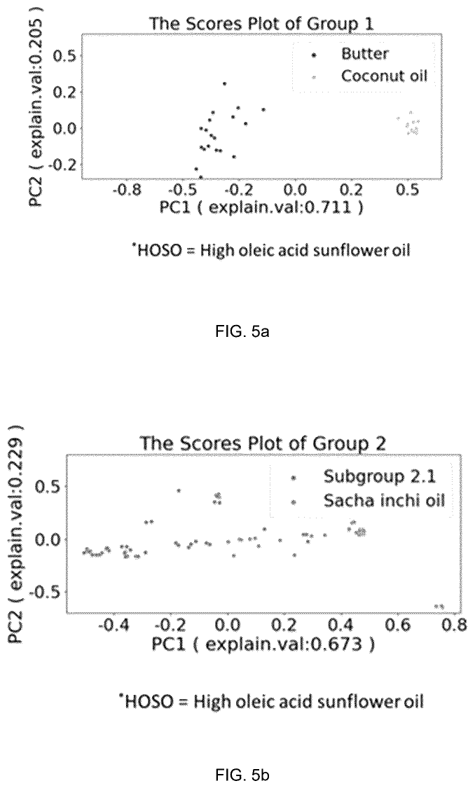

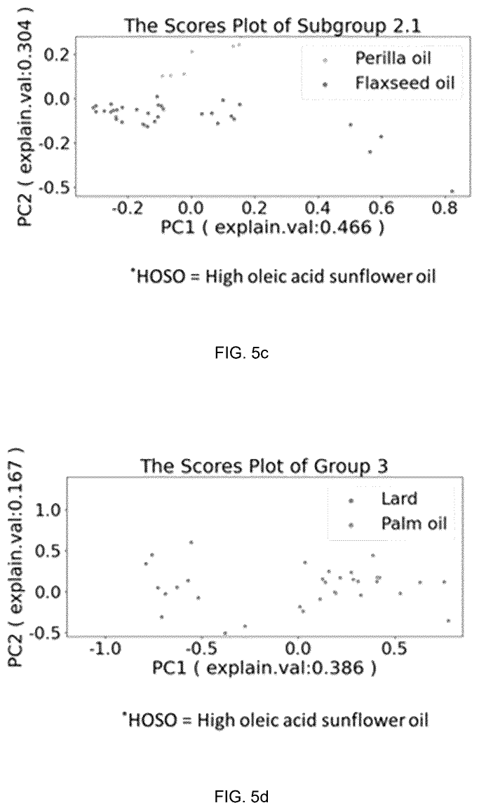

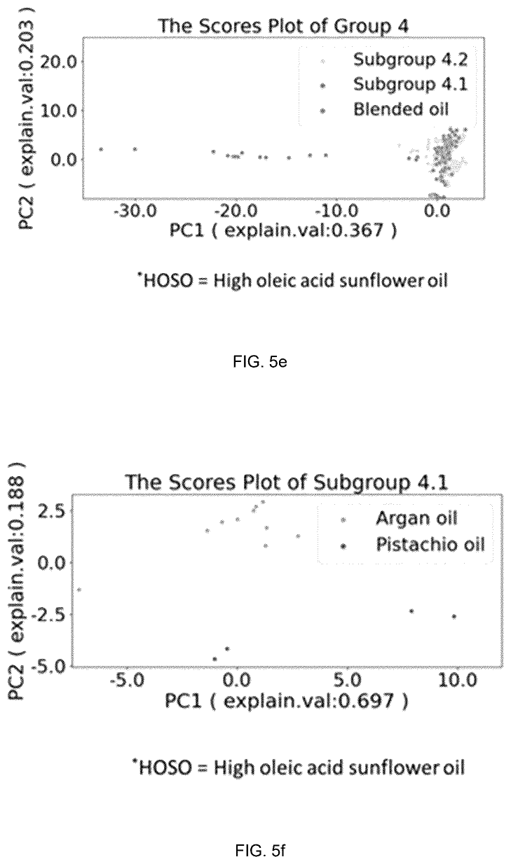

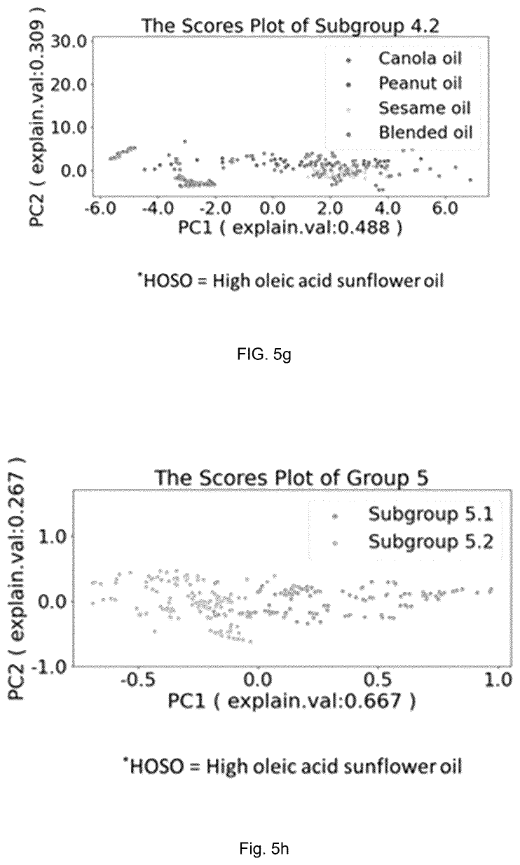

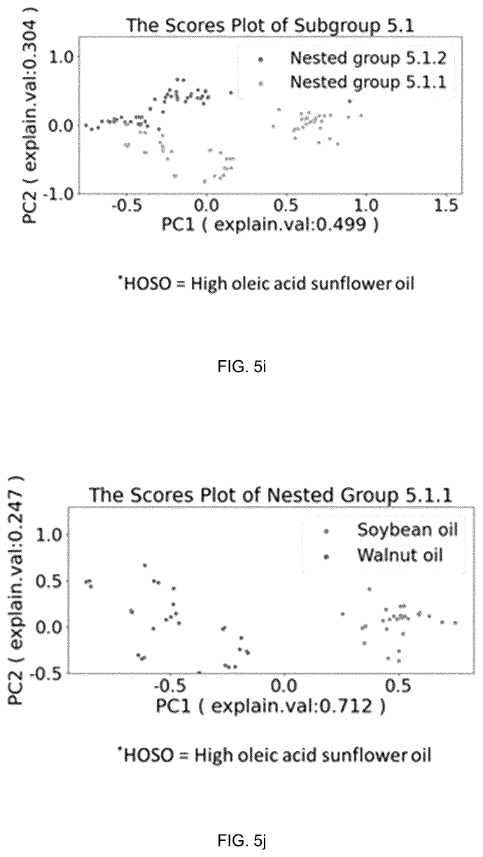

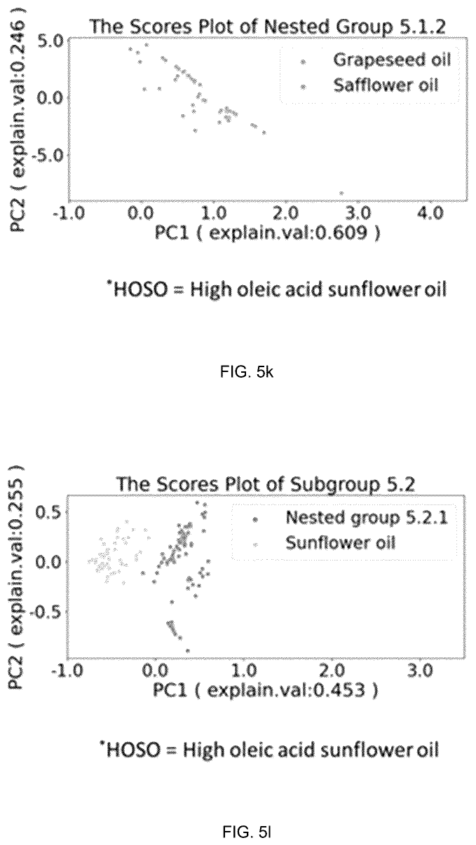

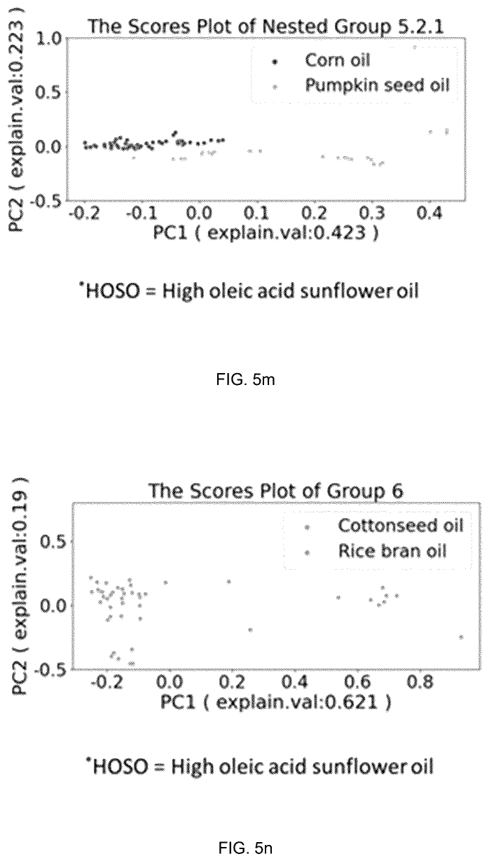

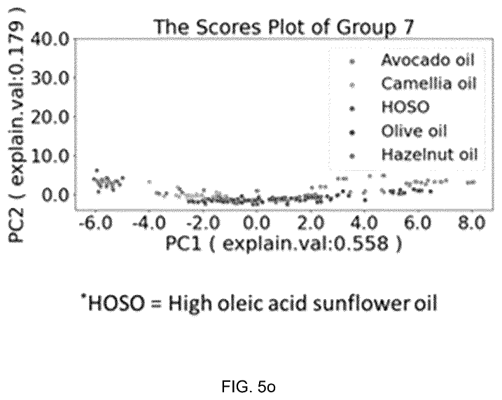

The edible oil types of the hierarchical library 14 are grouped by the normalized abundance differences according to the score plots obtained by Principal Component Analysis. FIG. 5a to FIG. 5o depict exemplary score plots obtained by Principal Component Analysis (PCA) for the grouping in the hierarchical library 14.

It should be noted that the TAG peaks of Group 8 have different distinctive TAG patterns from each other, therefore no further grouping is undertaken of the edible oil types of Group 8 by applying the Principal Component Analysis.

Each level of said binary tree may include two members, each member is either (a) an intermediate representative groups comprising edible oil types grouped by Principal Component Analysis of the TAG peaks or (b) a single edible oil type defined using a determined maxima function of the TAG peaks of the known edible oil members therein. The terminal level of the hierarchical library may include two or more known edible oil members.

The machine learning module 18 is trained to assign edible oil samples to one of the Group 1 to Group 7 with the highest similarity score as shown in FIG. 2 and further classify the edible oil samples into subgroups according to similarity scores/probabilistic scores of MALDI mass spectral (TAG peaks) of edible oil types as compared to the TAG peaks of the edible oil types of the hierarchical library 14.

Gaussian mixture model (GMM) algorithm and Logistic regression (LR) algorithm are used to calculate the likelihoods of samples as the MALDI mass spectral similarity scores/probabilistic scores when training so as to determine an allocation of an appropriate edible oil type.

In one embodiment, classifications of edible oil samples are performed by different degrees of binary classification steps using the similarity scores calculated by the Gaussian mixture (GMM) algorithm. For example, edible oil samples assigned to Group 2 are classified as sacha inchi oil or Subgroup 2.1, and samples assigned to Subgroup 2.1 are further binary classified as perilla oil or flaxseed oil. A similar logic is applied to the classification of edible oil samples assigned to Group 5.

After optimizing the classification branch for Group 7, classifications of edible oil samples are performed by comparing the similarity scores from the logistic regression (LR) algorithm for the binary classification of hazelnut oil or Subgroup 7.1 including olive oil, avocado oil, camellia oil, and high oleic acid sunflower oil, followed by a quaternary classification of the remaining samples using similarity scores from the Gaussian mixture (GMM) algorithm.

In other embodiment, the hierarchical library 14 further include blended oil types, and the logistic regression (LR) algorithm is also used for differentiation of blended oil and peanut oil or sesame oil.

In detail, the machine learning module 18 is trained based on the constructed hierarchical library 14. The OVO strategy is used by the integration with the binary decision tree algorithm, a tree-like structure to split data based on feature values and probabilistic modules based on Gaussian mixture (GMM) algorithm or logistic regression (LR) algorithm. The probabilistic modules are used to obtain the probability of an edible oil samples in a primary, intermediate representative groups and edible oil types.

As discussed above, the edible oil samples are partitioned into smaller groups using Principal Component Analysis. Also, the important variables (i.e., the m/z values with the largest magnitude in the PCA loading plot) are selected in the calculation of similarity scores.

The probabilistic modules may adopt a Gaussian mixture model (GMM) algorithm to calculate the probabilistic scores of samples for the classification of recorded edible oil types except peanut oil and sesame oil. This allows the calculation of the probabilistic scores of samples for the classification of recorded edible oil types except peanut oil and sesame oil.

The decision tree module is employed to determine the classification directions of edible oil samples by applying the maxima function to their similarity scores.

In another example, if taking account into blended oil, due to the high similarities in the TAG region between the MALDI mass spectra of blended oil and either peanut oil or sesame oil, a logistic regression (LR) algorithm may be optimized for calculating the similarity/probabilistic scores for the differentiation of blended oil and peanut oil or sesame oil.

A series of tree-based binary classifications of edible oil samples based on the decision maxima function to their calculated probabilistic score for the identification of samples is performed. The function of the maxima function is to return the maximum calculated probabilistic scores (r).

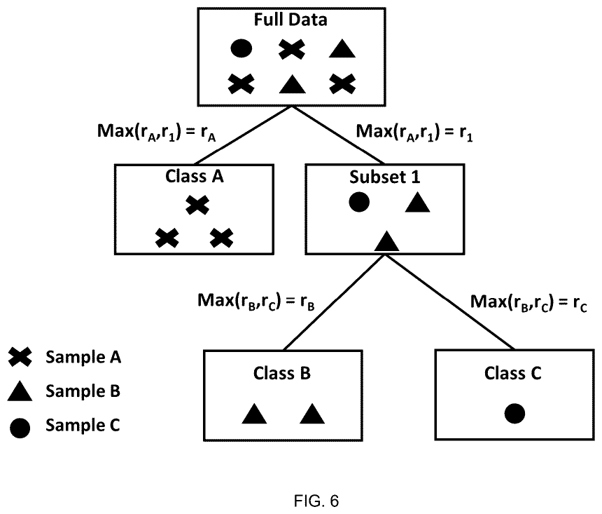

This process is depicted graphically in FIG. 6. As depicted above, the hierarchical library 14 is constructed based on the Principal Component Analysis results.

The structural organization of the hierarchical library 14 is directly to the algorithmic framework of the machine learning module 18. Specifically, the PCA (Principal Component Analysis)-derived groupings enable targeted partitioning through mathematically defined functions, as detailed below.

In one embodiment, the general maximum function (R(x)) is used to return the maximum value (see Equation (1)). The function (O(x)) is used to return the classified group corresponding to the maximum value (see Equation (2)) for the partition using the hierarchical library 14 in the machine learning module 18.

R(x)=max(A) (1)

O(x)=arg max(A) (2)

For the partition of class (i.e., primary, intermediate representative groups or edible oil types) in the hierarchical library 14 using the Gaussian Mixture Model algorithm, the matrix A and f (x) can be defined as Equation (3) and Equation (4) respectively.

$$A = ( \begin{matrix}f ( {x}_{1 , 1} ) & ⋯ & f ( {x}_{1 , n} ) \\ ⋮ & ⋱ & ⋮ \\ f ( {x}_{n , 1} ) & ⋯ & f ( {x}_{n , n} )\end{matrix} ) \qquad ( 3 )$$

$$f ( x ) = {\sum }_{k = 1}^{K} {π}_{k} N ( ( x \vert {μ}_{k} , {\sum }_{k} ) ) \qquad ( 4 )$$

Where:
   K is the number of class in the partition step
   πₖ is the mixing coefficient of the k^{th} class in the partition of hierarchical library therefore:

$${\sum }_{k = 1}^{K} {π}_{k} = 1 and 0 \leq {π}_{k} \leq 1 \qquad ( 5 )$$

N(x|μₖ, Σk) is the multivariate Gaussian distribution of the k^{th} class and defined as equ.6:

$$N ( x \vert {μ}_{k} , {\sum }_{k} ) = \frac{1}{{( 2 π )}^{n / 2} {\vert {\sum }_{k} \vert}^{1 / 2}} {e}^{( - \frac{1}{2} {( x - {μ}_{k} )}^{{T}_{{\sum }_{k}} - 1} ( x - {μ}_{k} ) )} \qquad ( 6 )$$

Where:
   μₖ is the mean vector of the normalized TAG peaks in the k^{th} class;
   Σₖ is the covariance matrix of the normalized TAG peaks of the k^{th} class;
   n is the number of the normalized TAG peaks;
   x is the input normalized TAG peaks of samples.

Since the TAG patterns of primary, intermediate representative groups or edible oil types in the hierarchical library are uniquely assigned based on the PCA analysis and further used to select the corresponding TAG peaks as variables in the calculation of mean vector and covariance matrix in Equation (6), the non-diagonal entries (i.e., multivariate normal distributions of the primary, intermediate representative groups or edible oil types with the unmatched TAG pattern peaks) considered as 0. Therefore, the matrix A could be further expressed as follows:

$$A = ( \begin{matrix}f ( {x}_{1 , 1} ) & ⋯ & 0 \\ ⋮ & ⋱ & ⋮ \\ 0 & ⋯ & f ( {x}_{n , n} )\end{matrix} ) \qquad ( 7 )$$

For the partition of primary, intermediate representative groups or edible oil types in the hierarchical library using the LR algorithm, the vector A and f (x) for Equation (3) can be defined as Equation (8), and Equation (9) respectively.

$$A = ( \begin{matrix}f ( Y = {k}^{th} Class ) \\ f ( Y \neq {k}^{th} Class )\end{matrix} ) \qquad ( 8 )$$

$$f ( x ) = \frac{1}{1 + {e}^{- t}} \qquad ( 9 )$$

Where:
Y is the possible class of samples in the partition step; and t is linear combination of the TAG peaks for the selected TAG pattern, and can be expressed as follows:

t=β₀+β₁x₁ . . . +βₙxₙ (10)
Where:
n is the number of TAG peak in the selected TAG pattern; x is the TAG peak in the selected TAG pattern; and β is the coefficient of x in the Logistic regression algorithm.

To identify unknown oil types, in the system 10, a series of tree-based binary classifications of edible oil samples are performed based on the decision of maxima function to their calculated probabilistic/similarity score for the identification of samples. The calculated probabilistic/similarity scores are used for the classification of edible oil samples.

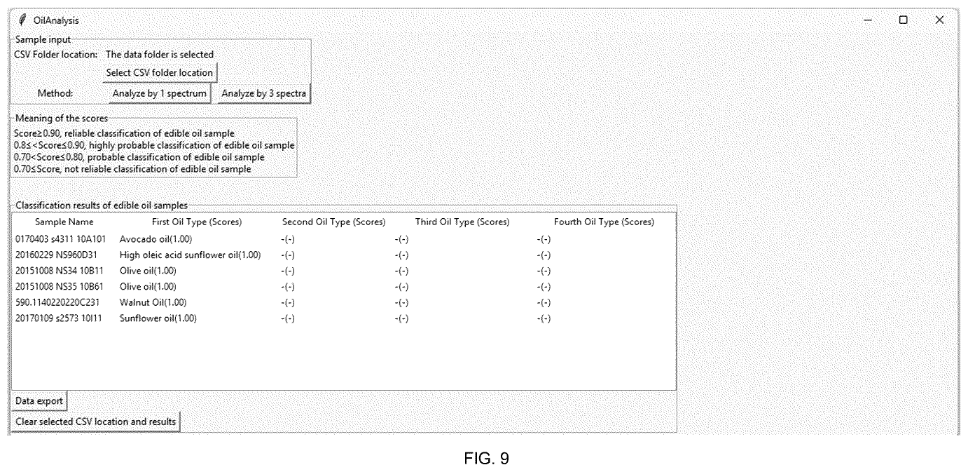

As discussed below by referring to FIG. 9, The calculated probabilistic scores are also used for estimating certainty level of the classification results by using testing dataset.

FIG. 7 illustrates an example of the simplified flow chart of the method of identifying an unknown edible oil sample of the present disclosure. The method starts at step S01.

At step S02, a hierarchical library of known edible oil samples is generated.

The generating of the hierarchical library of known edible oil samples includes classifying a plurality of edible oil samples having known types into a predetermined number of primary representative groups according to intensity of one or more characteristic TAG peaks for that group using PCA analysis. The generating of the hierarchical library of known edible oil samples may further include decomposing the primary representative groups using a binary tree classification network; wherein each level of said binary tree includes two members, said two members being either intermediate representative groups comprising edible oil types grouped by PCA analysis of the TAG peaks or a single edible oil type defined using a determined maxima function of the TAG peaks of the known edible oil members therein;

At step S03, spectral data including at least one or more TAG peaks for an unknown edible oil sample is determined using matrix-assisted laser desorption/ionization mass spectrometry (MALDI-MS).

At step S04, a machine learning module is trained to:—
(a) compare the obtained spectral data with TAG peaks for said unknown edible oil sample against the characteristic TAG peaks in each primary representative group of the hierarchical library of edible oil samples by calculating the similarity score between each primary representative group and the unknown edible oil sample;
(b) select the primary representative group with the highest similarity score from the plurality of primary representative groups; and
(c) compare the obtained spectral data with TAG peaks for said unknown edible oil sample by determining the similarity score between the obtained spectral data with TAG peaks for said unknown edible oil with the characteristic TAG peaks of the respective intermediate group; or the characteristic TAG peaks of the single representative oil type; and selecting either the intermediate group member or single representative edible oil type with the highest similarity score;
(d) repeat step (c) above until a presumptive single edible oil type for the unknown edible oil sample is identified.

The method finishes at step S05.

To train a machine learning module to analyse unknown edible oils, approaches for collecting data of sample and unknow edible oils and training the machine learning module are discussed below in detail.

The experiments and laboratory test are discussed below in detail.

The samples of argan oil, pistachio oil, macadamia oil, and sacha inchi oil are purchased from Hong Kong, Mainland China, and Taiwan. Three bottles for each commercially available edible oil product are purchased for triplicate measurements.

The blended oil samples are prepared in-house by mixing pure olive oil with pure canola oil, corn oil, peanut oil, and sunflower oil in binary combinations (i.e., 5%: 95%, 20%: 80%, 30%: 70%, 40%: 60%, and 50%: 50%).

All the samples are aliquoted and sealed in amber bottles and retained at room temperature and pressure before MALDI mass spectrometric analysis. MALDI matrices a-cyano-4-hydroxycinnanic acid (CHCA), 2,5-dihydroxybenzoic acid (DHB), trifluoroacetic acid, and sodium iodide are purchased from Sigma-Aldrich (St. Louis, MO, USA). HPLC-grade acetone and HPLC-grade acetonitrile are purchased from DUKSAN. Polyethylene glycol (PEG) standards are purchased from Fluka (Buchs, Switzerland). All chemicals are directly used without further purification.

Aliquots of around 0.5 μL of 100 mg mL⁻¹ DHB in acetone are pre-loaded onto spots of the MALDI plate and air-dried. Each edible oil sample is directly loaded as a thin layer on top of the dried DHB layer by using the medical cotton tip. Then the MALDI plate is inserted into the MALDI mass spectrometer for analysis.

In one embodiment, an ultrafleXtreme MALDI-TOF/TOF mass spectrometer equipped with a 355 nm smartbeam-II laser is used for the MALDI mass spectrometric analysis. The mass spectrometer is operated at a frequency of 2000 Hz, positive and reflectron mode for the analysis. The matrix suppression cut-off value is set to 400 Da, and the MALDI mass spectra are obtained within 500-2000 Da. The ion pulse excitation is achieved through 140 ns. The ion source voltage 1, ion source voltage 2, lens voltage, reflector voltage 1, reflect voltage, and reflector base detector voltage are set to 20 kV, 18 kV, 8.5 kV, 21.1 kV, 10.85 KV, and 1.8 kV, respectively. The sample rate is set to 5.0 GS/s. The calibration of the mass spectrometer is achieved using the PEG mixture (PEG600/PEG1000/PEG2000/NaI=1/2/2/5 v/v).

For the automatic spectral acquisitions of samples using AutoXecute in flexControl 3.4, the MS/Parent mode with the weight of 1 is used in the Fuzzy Control, and the random walk with 100 shots at the raster spot is adopted in the setting of the laser. In total, 7 spectra with a resolution higher than 3000 in the TAGs range (typically in the mass range of 870 to 910 Da) using 1000 shot steps are summed as final spectra with the absolute intensity of the highest peak reached a value of 2×10⁴ and saved as one spectrum for further analysis.

The acquired mass spectra are processed using the flexAnalysis 3.4 program (Bruker, Billerica, MA, USA), applying the “centroid” peak detection algorithm with a S/N of 4 and peak width of 0.2 Da, “TopHat” Baseline Subtraction Algorithm and “SavitzkyGolay” peak smoothing algorithm with a peak width of 0.2 Da and 1 cycle. Each edible oil sample is analyzed in triplicate to ensure the consistency of the detected peaks. The spectral data including the m/z, absolute intensities, and S/N of edible oil samples are automatically exported to the “.csv” file format for the statistical analysis and algorithmic analysis in OilAnalysis by employing the SBA script to the “Marco editor” in the flexAnalysis 3.4.

It would be appreciated that the identification of the detected phospholipids, peptides, and sodium-adducted TAGs are known by decrypting the detected peaks in the MALDI-MS spectra.

For example, the identification of the detected phospholipids was discussed by Calvano, C. D., et al., in 2012 (see “MALDI-TOF mass spectrometry detection of extra-virgin olive oil adulteration with hazelnut oil by analysis of phospholipids using an ionic liquid as matrix and extraction solvent”, Food Chemistry, 134(2), 1192-1198) and by Stefanowicz, P. in 2001 (see “Electrospray Mass Spectrometry and Tandem Mass Spectrometry of the Natural Mixture of Cyclic Peptides from Linseed”, European Journal of Mass Spectrometry, 10(5), 665-671); the identification of the detected peptides is discussed by is discussed by Lay, J. O., et al., in 2006 (see “Rapid characterization of edible oils by direct matrix-assisted laser desorption/ionization time-of-flight mass spectrometry analysis using triacylglycerols”, Rapid communications in mass spectrometry, 20(6), 952-958); and the identification of the detected sodium-adducted TAGs is discussed by Schiller, J., et al., in 2004 (see “Matrix-assisted laser desorption and ionization time-of-flight (MALDI-TOF) mass spectrometry in lipid and phospholipid research”, Progress in Lipid Research, 43(5), 449-488).

The exported data are aligned and normalized by the chosen peaks, followed by the average of the triplicated samples. However, the ions below m/z 630 are not included in the chosen peaks list because of the interferences from the possible DAG-like ions within m/z 600 to m/z 630 (Al-Saad et al., 2003; Gidden et al., 2007). The processed data of the pure edible oil type are input into the statistics software Umetrics Simca (v. 16.0, Andover, MA, USA) for the analysis using PCA models. The settings of raw PCA models are constructed using the “Autofit” mode and default setting.

For statistical analysis of the processed data of blended oils, the packages of NumPy and scikit-learn in Python 3.9 are used.

It should be understood that persons skilled in the art could use similar instruments and software for implementing the disclosed data collecting and processing.

The edible oil types of the samples are grouped by the normalized abundance differences according to the score plots (as shown in FIG. 5a to FIG. 5o) obtained by Principal Component Analysis. The results from the Principal Component Analysis reflect the major characteristics in the MALDI-MS spectra of the edible oil types of the samples.

For example, cottonseed oil and rice bran oil in Group 6 are differentiated by applying one time of principal component analysis. According to the score plots from principal component analysis, the cluster of the cottonseed oil and rice bran oil is clearly separated.

As depicted in FIG. 5a, FIG. 5d and FIG. 5n, from the Principal Component Analysis, the edible oil types Group 1 (including butter and coconut oil), Group 3 (including palm oil and lard), and Group 6 (including cottonseed oil and rice bran oil) are distinguishable by the normalized abundance differences according to their score plots

Groups 2, 4 and 5 consist of more than two types of edible oils. Therefore, multiple Principal Component Analysis are utilized. For example, for Group 2, one Principal Component Analysis is executed so as to separate sacha inchi oil, and another Principal Component Analysis is further executed to differentiate flaxseed oil and perilla oil.

Similar approaches are applied in the differentiation of edible oil types in Group 5.

As depicted in FIG. 5e, FIG. 5f and FIG. 5g, edible oil samples in Group 4 are subdivided into Subgroup 4.1 and Subgroup 4.2. From the score plots obtained by the Principal Component Analysis the argan oils and pistachio oils in Subgroup 4.1 and the cluster canola oil, peanut oil, and sesame oil in Subgroup 4.2 are distinctively recognizable.

In this embodiment, principal component analysis only aids the identification of high oleic acid sunflower oil and no partition is applied between hazelnut oil and Subgroup 7.1 in the score plot from their PCA result, as no distinct separation between their clusters is observed (see FIG. 5o). However, the logistic regression algorithm may be used to partition them because it is a probability-based binary classification model.

Castor oil, fish oil, and macadamia oil having signature TAG patterns are classified as Group 8. As TAG peaks of Group 8 have distinctive TAG patterns when compared with each other, Principal Component Analysis is not used to partition such oil types.

In one embodiment, the number of edible oils in the MALDI mass spectral database is increased to 1749. Also, the total number of edible oil types in the database is increased to 35.

It is noted that wheat germ oil, palm super olein oil, almond oil, or pine nut oil is rare in the market so these oils are excluded to make the modeling processing concise. The rapeseed oil type is combined with the canola oil type in algorithm construction because of their similar MALDI mass spectrometric patterns of major TAGs.

The nut type oils such as argan oils, pistachio oils, macadamia oils and sacha inchi oils are also included for analyzing by MALDI mass spectrometry. In the TAG region of their MALDI mass spectra, argan oils, pistachio oils, and macadamia oils are with ions of m/z 905.7 and m/z 907.7 as the highest peaks in the mass spectra, while sacha inchi oils is with ions of m/z 899.7 as the highest peak in the mass spectra. This difference allows the differentiation of these nut oils.

Further, the intensity of the peak at m/z 853.7 for macadamia oil is significantly higher than for argan oil and pistachio oil, allowing the differentiation of macadamia oil. High intensities of peaks, which are assigned as phospholipids, such as m/z 760.6 ([PC(C16:0/C18:1)+H]+), m/z 786.6 ([PC(C18:1/C18:1)+H]+) and m/z 760.6 ([PC(C18:0/C18:2)+H]+), are present in the mass spectra of argan oil, pistachio oil and macadamia oil. These peaks are likely attributed to the absence of degumming and the adoption of the cold-pressing technique in the production of edible oils.

Before training the machine learning module, the dataset is trimmed to the dataset (N=1567) by removal of the anomalous samples (i.e., the samples having different TAGs patterns with the majority of that claimed edible oil type) and the trimmed dataset is further broken down to the training dataset (N=1024) and validation dataset (N=543).

The edible oil samples in the training dataset (N=1024) are divided into 8 groups shown in FIG. 2. Castor oil, fish oil, and macadamia oil having signature TAG patterns are classified as Group 8. While sacha inchi oil is assigned to Group 2, argan oil, and pistachio oil are assigned to Group 4 due to their major abundant peaks in the TAGs region.

In a further aspect, a machine learning module is constructed for the calculation and evaluation of similarity scores based on the training dataset to classify the edible oil samples.

The classification approach is based on principal component analysis (PCA) to differentiate edible oil types and to further identify their normalized abundance differences for the calculation of similarity scores in the machine learning mode.

For the classification steps in the machine learning module, a decision tree is employed to determine the classification directions of edible oil samples by applying the maxima function to their similarity scores.

Generally, the Gaussian mixture algorithm is used to calculate the similarity scores for the differentiation of groups and edible oil types except for peanut oil and sesame oil.

It is known that TAG regions of blended oils often demonstrate complete overlap with peanut oils and sesame oils, indicating the high MALDI mass spectral similarity between the blended oils and either peanut oils or sesame oils. This similarity may temporarily group the blended oils in the cluster of peanut oil or sesame oil by principal component analysis, but these may be further differentiated from peanut oils or sesame oils by the adoption of a binary differentiation step based on the Logistic regression (LR) algorithm not the Gaussian mixture algorithm. The Logistic regression algorithm is used to the differentiation of blended oil, peanut oil, and sesame oil respectively.

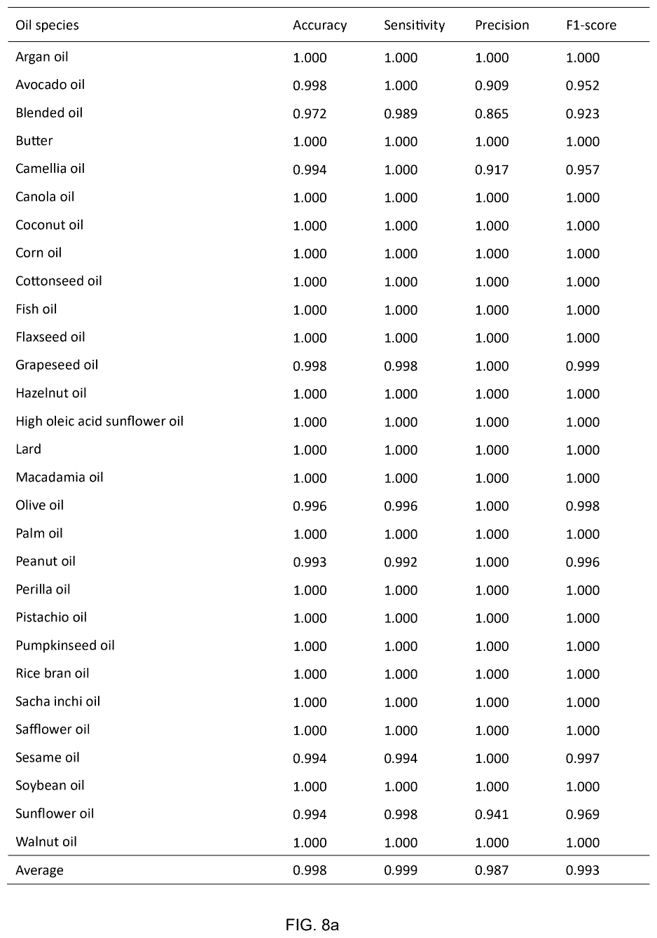

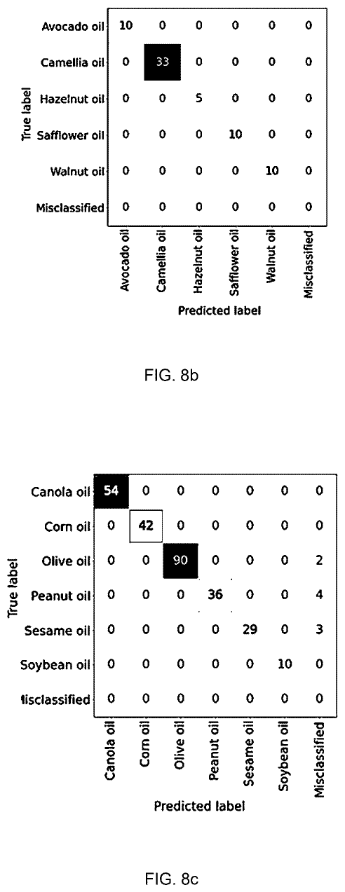

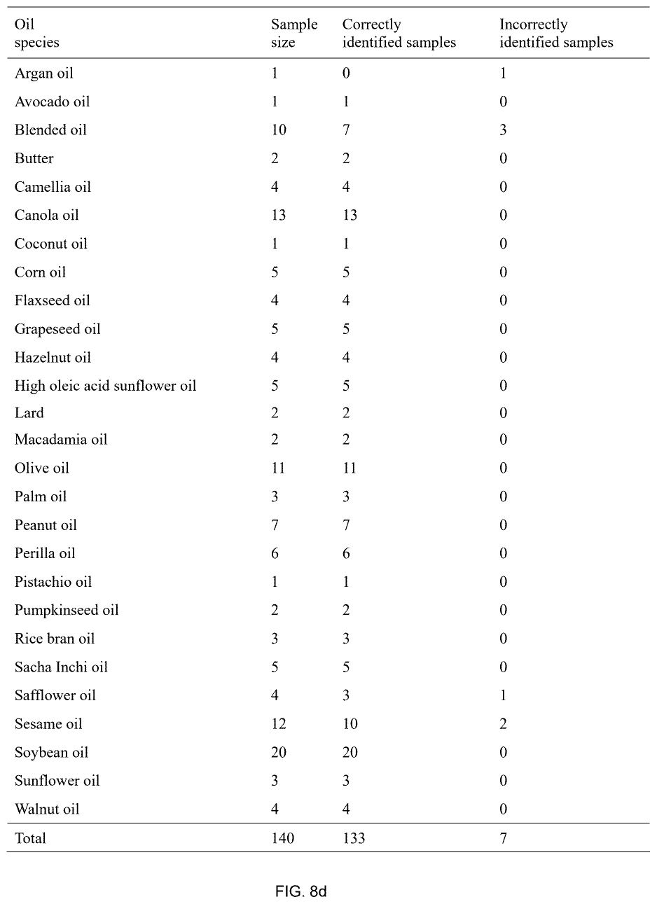

The Gaussian mixture (GMM) algorithm and Logistic regression (LR) algorithm could also be used to quantify the uncertainty of prediction/identification as discussed below by referring FIG. 8.

The normalized abundances used for differentiations of edible oil samples in principal component analysis are selected as the variable sets to calculate the similarity scores in the Gaussian mixture algorithm for the classification of edible oils in the machine learning module. For the variable sets used in the logistic regression algorithm to differentiate the samples of blended oil and samples of peanut oil and sesame oil in the machine learning module, variables having relative standard deviation <30 are selected.

The dataset from the database (N=1567) is divided to training dataset (N=1024) and validation dataset (N=543). The testing dataset (N=140) is obtained from a collection of recently measured samples rather than the database.

Overall, a correct classification rate of 97.9% is achieved, with 21 samples incorrectly classified as other edible oil types in the machine learning module using the training dataset. Also, machine learning module correctly classifies 97.8% (349 out of 358 samples) of common edible oils (i.e., olive oil, canola oil, soybean oil, peanut oil and sesame oil), 98.9% (88 out of 89 samples) of rare edible oils (i.e., walnut oil, sacha inchi oil, argan oil, pistachio oil, and macadamia oil), and 98.4% (125 out of 126 samples) of blended oil.

The validation dataset (N=543) is used to evaluate the performance of the machine learning module.

It obtains from a collection of recently measured samples about testing data (N=140) with 27 edible oil types obtained from the autoflex MALDI-TOF/TOF mass spectrometer is used to test the performance of the trained machine learning module.

FIGS. 8a to 8c show the performance of the trained machine learning module regarding the validation data and FIG. 8d shows the identification results obtained by the trained machine learning module regarding the testing data.

Precision, accuracy, sensitivity, and F1-score are employed to evaluate positive prediction accuracy, overall correctness, correct sample prediction ability, and balance between precision and sensitivity to the model, respectively.

The calculation of the precision, accuracy, sensitivity, and F1-score is provided below: A set of edible oil types (A) is used to represent each edible oil sample (x) as follows:

Aₓ={CEₓ,PEₓ}

Where CE is the claimed edible oil type of samples and PE is the predicted edible oil type.

The set of true positive (TP), false positive (FP), true negative (TN), and false negative (FN) for the classification of each edible oil type (RET) recorded in the database is defined as follows:

TP={CEₓ=RET∧PEₓ=RET}

FP={CEₓ≠RET∧PEₓ=RET}

TN={CEₓ≠RET∧PSₓ≠RET}

FN={CEₓ=RET∧PSₓ≠RET}

The accuracy, sensitivity, precision and F1-score for the classification of each edible oil type are calculated based on the equations (10) to (13) listed below:

$$Accuracy = \frac{\vert TP \vert + \vert TN \vert}{\vert TP \vert + \vert FP \vert + \vert TN \vert + \vert FN \vert} \qquad ( 10 )$$

$$Sensitivity = \frac{\vert TN \vert}{\vert TN \vert + \vert FN \vert} \qquad ( 11 )$$

$$Precision = \frac{\vert TP \vert}{\vert TP \vert + \vert FP \vert} \qquad ( 12 )$$

$$F 1 - score = \frac{2 \times Precision \times sensitivity}{Precision + Sensitivity} \qquad ( 13 )$$

Where |TP|, |FP|, |TN| and |FN| are the number of elements in the set of TP, FP, TN and FN respectively.

When precision, accuracy, sensitivity, and F1-score all have the value of 1, the classification model is considered perfect.

As shown in FIG. 8a, the average accuracy, sensitivity, precision, and F1-score of the machine learning module of the present disclosure are 0.998, 0.999, 0.987, and 0.993 respectively. This result indicated that the machine learning module is effective in predicting the true edible oil types of samples in the validation dataset which made it a robust and reliable model for the classification of edible oils in the database.

The performance of the machine learning module can be also visualized using the confusion matrix as shown in FIG. 8b and FIG. 8c. Actual/true class labels are presented as rows and predicted class labels as columns. When the confusion matrix is the diagonal matrix (i.e., a square matrix with all zero in off-diagonal entries), the classification performance of the machine learning module is considered perfect because the predicated labels of samples are the same as their true labels. In contrast, the mis-classified samples are indicated by non-zero entries in off-diagonal positions of the confusion matrix.

For the performance in the classification of common edible oils, the machine learning module misclassified 9 samples (see FIG. 8c). Regarding the performance in classifications of rare edible oils, the machine learning module correctly classified all samples (see FIG. 8b). FIG. 8b shows the diagonal confusion matrix, showing that the machine learning module correctly classifies all the rare edible oil samples.

In general, the machine learning module of the present disclosure demonstrates higher average accuracy, precision, and sensitivity than other models in the field in the identification of edible oils in the usage of an imbalanced training dataset. The machine learning module of the present disclosure can more accurately identify the samples to its true edible oil type.

Overall, as shown in FIG. 8d, 95% (133 out of 140) of testing samples are correctly identified using the system of the present disclosure, indicating the high accuracy of the system 10 in predicting the data external to the algorithm database.

The trained machine learning module of the present disclosure may be configured to couple with for example flexAnalysis 3.4 to be developed to enable direct analysis of spectral data from MALDI mass spectrometry and exporting corresponding identification results. It should be understood that other programs may also be available, without departing from the scope of the present disclosure.

FIG. 9 depicts an exemplary user interface for an embodiment of the system of the present disclosure for identifying an unknown edible oil sample. The results of using the system and method of the present disclosure is depicted; with six unknown samples shown, all of which were identified as comprising the highly probable oil types noted withs score of 1. In this case, it was not necessary for second, third or fourth oil type scores. However, as shown, it would be appreciated that the system 10 is also able to label the suspicious samples with a lower or no similarity scores, showing the reliability of the trained machine learning module 18 in the correct identification of edible oil samples.

It has been observed that in an embodiment of the present disclosure, the system 10 can process 60 spectra within 35 seconds (1.7 spectra per second), resulting in a substantial reduction in analysis time for large sample sizes, and hence offering high throughput and practical applications.

The system of the present disclosure is simple and user-friendly and can be operated without needing expert knowledge. The system 10 may be incorporated with other commercially available software for software for the analysis of MALDI mass spectra as an alternative to flexAnalysis 3.4. Accordingly, the system provides high-throughput edible oil analysis with automatic data preprocessing of raw MALDI mass spectrometric data, data analysis using machine learning approach, result interpretation and display.

The Gaussian Mixture Module (GMM) algorithm and Logistic regression (LR) algorithm could also be used to quantify the uncertainty of prediction/identification, providing “Scores” as shown in FIG. 9. From the testing of the machine learning module, if a score is ≥0.9, then the classification of edible oil sample is reliable; if a score is >0.8 and ≤0.9, then the classification of edible oil sample is highly probable; if a score is >0.7 and ≤0.8, then the classification of edible oil sample is probable; if a score is ≤0.7 then the classification of edible oil sample is not reliable.

The above embodiments are described by way of example only. Many variations are possible without departing from the scope of the disclosure as defined in the appended claims.

For clarity of explanation, in some instances the present technology may be presented as including individual functional blocks including functional blocks comprising devices, device components, steps or routines in a method embodied in software, or combinations of hardware and software.

Methods according to the above-described examples can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, Universal Serial Bus (USB) devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information is used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

1. A method of identifying an unknown edible oil sample comprising:
generating a hierarchical library of known edible oil samples, by classifying a plurality of edible oil samples having known types into a predetermined number of primary representative groups according to intensity of one or more characteristic TAG peaks for that group using PCA analysis;
further decomposing the primary representative groups using a binary tree classification network; wherein each level of said binary tree includes two members, said two members being either intermediate representative groups comprising edible oil types grouped by PCA analysis of the TAG peaks or a single edible oil type defined using a determined maxima function of the TAG peaks of the known edible oil members therein and the terminal level of the hierarchical library has two or more known edible oil members;
determining using matrix-assisted laser desorption/ionization mass spectrometry MALDI-MS spectral data including at least one or more TAG peaks for an unknown edible oil sample;
training a machine learning module to:
(a) compare the obtained spectral data with TAG peaks for said unknown edible oil sample against the characteristic TAG peaks in each primary representative group of the hierarchical library of edible oil samples by calculating the similarity score between each primary representative group and the unknown edible oil sample;
(b) select the primary representative group with the highest similarity score from the plurality of primary representative groups; and
(c) compare the obtained spectral data with TAG peaks for said unknown edible oil sample by determining the similarity score between the obtained spectral data with TAG peaks for said unknown edible oil with the characteristic TAG peaks of the respective intermediate group; or the characteristic TAG peaks of the single representative oil type; and selecting either the intermediate group member or single representative edible oil type with the highest similarity score;
(d) repeat step (c) above until a presumptive single edible oil type for the unknown edible oil sample is identified.

2. The method of claim 1, wherein the obtained spectral data with TAG peaks for said unknown edible oil sample and the spectral data of the hierarchical library of edible oil samples are normalized.

3. The method of claim 1, wherein the hierarchical library of known edible oil samples has eight representative groups.

4. The method of claim 1 wherein said eight representative groups comprise edible oils with TAG peaks of a first group comprising 659.6, 660.6, 687.6, 688.6; TAG peaks of a second group comprising 895.7, 897.7, 899.7, TAG peaks of a third group comprising 853.7, 855.7, 879.8, 880.8, 881.8, TAG peaks of a fourth group comprising 880.8, 881.8, 903.8, 904.8, 905.8, 906.8, 907.8, 908.7, TAG peaks of a fifth group comprising 901.8, 902.8, 903.8, 904.8, 905.8, 906.8, 907.8, TAG peaks of a six group comprising 877.8, 878.8, 879.8, 880.8, TAG peaks of a seventh group comprising 853.7, 854.7, 879.8, 880.8, 881.8, 882.8, 905.8, 906.8, 907.8, 908.7, 909.7, TAG peaks of a eighth group comprising 735.3, 806.6, 828.6, 927.8, 955.7, 956.7, 957.7, 981.7, 1019.5, 1045.5

5. The method of claim 1, wherein the similarity scores are calculated based on Gaussian mixture algorithm or Logistic regression algorithm.

6. A system for identifying an unknown edible oil sample comprising:
a data store comprising a hierarchical library of known edible oil samples, said library including a plurality of edible oil samples classified into known types into a predetermined number of primary representative groups according to intensity of one or more characteristic TAG peaks for that group using PCA analysis; wherein said primary representative groups are further decomposed using a binary tree classification network; wherein each level of said binary tree includes two members, said two members being either intermediate representative groups comprising edible oil types grouped by PCA analysis of the TAG peaks or a single edible oil type defined using a determined maxima function of the TAG peaks of the known edible oil members therein and the terminal level of the hierarchical library has two or more known edible oil members;
a processor configured for comparing the matrix-assisted laser desorption/ionization mass spectrometry (MALDI-MS) spectral data including at least one or more TAG peaks for an unknown edible oil sample against the characteristic TAG peaks in each primary representative group of the hierarchical library of edible oil samples by a machine learning module;
wherein the machine learning module is trained to
(a) compare the obtained spectral data with TAG peaks for said unknown edible oil sample against the characteristic TAG peaks in each primary representative group of the hierarchical library of edible oil samples by calculating the similarity score between each primary representative group and the unknown edible oil sample;
(b) select the primary representative group with the highest similarity score from the plurality of primary representative groups; and
(c) compare the obtained spectral data with TAG peaks for said unknown edible oil sample by determining the similarity score between the obtained spectral data with TAG peaks for said unknown edible oil with the characteristic TAG peaks of the respective intermediate group; or the characteristic TAG peaks of the single representative oil type; and select either the intermediate group member or single representative edible oil type with the highest similarity score;
(d) repeat step (c) above until a presumptive single edible oil type for the unknown edible oil sample is identified.

7. The system of claim 6, wherein the obtained spectral data with TAG peaks for said unknown edible oil sample and the spectral data of the hierarchical library of edible oil samples are normalized.

8. The system of claim 6, wherein the hierarchical library of known edible oil samples has eight representative groups.

9. The system of claim 6, wherein said eight representative groups comprise edible oils with TAG peaks of a first group comprising 659.6, 660.6, 687.6, 688.6; TAG peaks of a second group comprising 895.7, 897.7, 899.7, TAG peaks of a third group comprising 853.7, 855.7, 879.8, 880.8, 881.8, TAG peaks of a fourth group comprising 880.8, 881.8, 903.8, 904.8, 905.8, 906.8, 907.8, 908.7, TAG peaks of a fifth group comprising 901.8, 902.8, 903.8, 904.8, 905.8, 906.8, 907.8, TAG peaks of a six group comprising 877.8, 878.8, 879.8, 880.8, TAG peaks of a seventh group comprising 853.7, 854.7, 879.8, 880.8, 881.8, 882.8, 905.8, 906.8, 907.8, 908.7, 909.7, TAG peaks of a eighth group comprising 735.3, 806.6, 828.6, 927.8, 955.7, 956.7, 957.7, 981.7, 1019.5, 1045.5.

10. The system of claim 6, wherein the similarity scores are calculated based on Gaussian mixture algorithm or Logistic regression algorithm.

11. A computer program product for identifying an unknown edible oil sample which when executed by a processor causes it to perform the method of claim 1.
